(19) United States
(12) Patent Application Publication
(10) Pub. No.: US 2025/0297295 A1
(43) Pub. Date: Sep. 25, 2025

(54) PREPARATION METHOD OF PECTIC POLYSACCHARIDE CAPABLE OF REGULATING AND CONTROLLING ICE CRYSTAL GROWTH

(71) Applicant: INSTITUTE OF FOOD SCIENCE AND TECHNOLOGY, CHINESE ACADEMY OF AGRICULTURAL SCIENCES, Beijing (CN)

(72) Inventors: Jinfeng BI, Beijing (CN); Jianyong YI, Beijing (CN)

(73) Assignee: INSTITUTE OF FOOD SCIENCE AND TECHNOLOGY, CHINESE ACADEMY OF AGRICULTURAL SCIENCES, Beijing (CN)

(21) Appl. No.: 19/231,542

(22) Filed: Jun. 8, 2025

Related U.S. Application Data
(63) Continuation-in-part of application No. 18/896,834, filed on Sep. 25, 2024.

(30) Foreign Application Priority Data
Dec. 1, 2023 (CN) 202311636476.X

(51) Int. Cl. C12P 19/04; C12P 19/14; C12P 19/16

(52) U.S. Cl. CPC C12P 19/04; C12P 19/14; C12P 19/16

(57) ABSTRACT

The present disclosure provides a preparation method of a pectic polysaccharide capable of regulating and controlling ice crystal growth, comprising step 1, extracting a pectin crude extract from apple powder; step 2, carrying out branched chain enzymolysis on pectin crude extract to obtain pectin enzymatic hydrolysate; step 3, adding pectin methylesterase and pectin acetylesterase into pectin enzymatic hydrolysate for precise de-esterification enzymolysis; step 4, adding 0.5-2 U/mL of polygalacturonase into pectin enzymatic hydrolysate obtained in step 3, and hydrolyzing for 1.5-2.5 hours; and step 5, collecting precipitate in pectin enzymatic hydrolysate obtained in step 4 to obtain pectic polysaccharide. The pectic polysaccharide has a purity higher than 90%, a methyl esterification degree of 45-55%, an acetylation degree less than 2%, a neutral sugar content less than 5%, and an average molecular weight of 1.4-7.5 kDa, and obviously reduces an ice crystal size in a freezing process.

### CROSS-REFERENCE TO RELATED APPLICATION

This application is a continuation-in-part application of and claims the priority benefit of U.S. application Ser. No. 18/896,834, filed on Sep. 25, 2024, which claims the priority benefit of China application serial no. 202311636476.X, filed on Dec. 1, 2023. The entirety of each of the above-mentioned patent applications is here by incorporated by reference herein and made a part of this specification.

### BACKGROUND

### Technical Field

The present disclosure belongs to the technical field of food processing and manufacturing, and relates to a preparation method of a pectic polysaccharide capable of regulating and controlling ice crystal growth.

Frozen and freeze-dried foods have developed rapidly in recent years and become important food categories. However, large ice crystals are easy to form in a freezing process of fruits and vegetables, which leads to a large amount of juice loss after thawing of quick-frozen fruits and vegetables, resulting in a decline in quality of the quick-frozen fruits and vegetables; or pore structures of the freeze-dried product are uneven, especially the formation of some large ice crystals will lead to the formation of large pores in the freeze-dried food, which will affect an overall crisp taste of the product.

It is found that some anti-freezing proteins extracted from animals and plants in cold regions and deep seas have a good effect on inhibiting ice crystal growth, but these anti-freezing proteins are expensive to prepare and cannot be applied to production on a large scale. In addition, recent studies have found that some polysaccharides with special structures also have obvious effects of inhibiting ice crystal growth, comprising carrageenan and microcrystalline cellulose. Pectin is one of the most widely available polysaccharides in nature. However, the extracted natural polysaccharide has poor ice crystal inhibiting effect and cannot meet the requirements as an anti-freezing additive. An inhibiting effect of the natural pectin on ice crystals is not obvious, where the reason may be that there are too many neutral sugar branches, which form steric hindrance that is not beneficial to the adsorption of pectin to a surface of the ice crystal, and meanwhile, excessive or unreasonable ester groups will also affect interaction between the pectin and the ice crystal. The present disclosure is intended to prepare a pectin molecule with high ice crystal growth inhibition activity.

### SUMMARY

The present disclosure solves at least the above problems and/or deficiencies and to provide at least the advantages that will be described later.

Based on the principle that polysaccharide of a specific structure has the effect of inhibiting ice crystal growth, the structure of the polysaccharide is modified and modified by using various physical and chemical means, so that the effect of inhibiting ice crystal growth is improved, and a preparation method of a pectic polysaccharide capable of regulating and controlling ice crystal growth is provided, which prepares a pectin molecule with high ice crystal growth inhibition activity. The pectin polysaccharide may be used for regulating and controlling ice crystal in production processes of quick-frozen fruits, quick-frozen aquatic products, freeze-dried food and the like.

To this end, a technical solution provided by the present disclosure is as follows.

A preparation method of a pectic polysaccharide capable of regulating and controlling ice crystal growth, comprises the following steps of:
step 1, extracting a pectin crude extract from apple powder: adding water to apple powder to obtain an apple solution, keeping the apple solution at a temperature of 94-97° C. for 1-5 minutes, cooling the apple solution, sequentially carrying out enzymolysis with α-amylase, protease, amyloglucosidase and cellulase to obtain an apple solution enzymatic hydrolysate, wherein a concentration of the α-amylase is 460-530 U/mL, a pH of the α-amylase is 5.5-6.5, a temperature is 55-70° C., and an enzymolysis time is 1 hour; a concentration of the protease is 56.7-64.5 U/mL, a pH of the protease is 7-7.5, a temperature is 50-65° C., and an enzymolysis time is 1 hour; a concentration of the amyloglucosidase is 285-310 U/mL, a pH of the amyloglucosidase during enzymolysis is 3.8-4.5, a temperature is 55-65° C., and an enzymolysis time is 1 hour; and a concentration of the cellulase is 18-25 U/mL, a pH of the cellulase during enzymolysis is 4.5-5.5, a temperature is 45-55° C., and an enzymolysis time is 2 hours;
keeping the apple solution enzymatic hydrolysate at a temperature of 94-97° C. for 1-5 minutes;
concentrating the apple solution enzymatic hydrolysate, and then carrying out the vacuum filtration,
wherein the vacuum filtration treatment comprises the following steps:
adding 3-8 times the weight of 95% (v/v) ethanol into the concentrated solution of the apple solution enzymatic hydrolysate, stirring uniformly and performing first vacuum filtration, then collecting solid-phase filter residues of the first vacuum filtration and adding ethanol with the same weight as that of the first vacuum filtration, performing second vacuum filtration, collecting solid-phase filter residues of the second vacuum filtration to dissolve in 25-30 times the weight of acetone, standing for 4-8 minutes, performing third vacuum filtration, then collecting solid-phase filter residues of the third vacuum filtration for hot acid water extraction to obtain a pectin crude extract, wherein an extraction pH is 2-3, an extraction time is 2-10 hours, a temperature of water is 65° C.-90° C., and an acid is 1 mol/L hydrochloric acid or 1 mol/L citric acid;
step 2, carrying out branched chain enzymolysis on the pectin crude extract obtained in the step 1, specially comprising: adding rhamnogalacturonase with a concentration of 2-5 U/mL into the pectin crude extract, adjusting a pH to be 3.5, subjecting for enzymolysis for 1-1.5 hours under a condition of 45° C. to obtain a first pectin enzymatic hydrolysate; adding galacturonidase and arabinosidase both with a concentration of 2-5 U/mL into the first pectin enzymatic hydrolysate, adjusting the pH to be 4.5, and subjecting for enzymolysis for 1-2 hours under a condition of 40° C. to obtain a second pectin enzymatic hydrolysate; and adding xylogalacturonidase with a concentration of 2-5 U/mL into the second pectin enzymatic hydrolysate, adjusting the pH to be 6.0, and subjecting for enzymolysis continuously for 2-2.5 hours under a condition of 50° C. to obtain a third pectin enzymatic hydrolysate, heating the third pectin enzymatic hydrolysate to 90° C., keeping the temperature for 10-30 seconds, and then carrying out enzyme deactivation;
step 3, adding pectin methylesterase and pectin acetylesterase into the third pectin enzymatic hydrolysate obtained in the step 2 after the enzyme deactivation for precise de-esterification enzymolysis to obtain a fourth pectin enzymatic hydrolysate, heating the fourth pectin enzymatic hydrolysate to 90° C., keeping the temperature for 10-30 seconds, and then carrying out enzyme deactivation, wherein a dosage of the pectin methylesterase is 0.2-2 U/mL, a dosage of the pectin acetylesterase is 0.1-0.2 U/mL, a precise de-esterification enzymolysis time is 1.0-2.5 hours, the pectin methylesterase is extracted from tomato, orange, pawpaw, broccoli, apple or carrot, a pH value the precise de-esterification enzymolysis is 5.8, and a temperature of the precise de-esterification enzymolysis is 45° C.;
step 4, adding 0.5-2 U/mL of polygalacturonase into the fourth pectin enzymatic hydrolysate obtained in the step 3 after the enzyme deactivation, and hydrolyzing for 1.5-2.5 hours to obtain a fifth pectin enzymatic hydrolysate to reduce a molecular weight of pectin, and heating the fifth pectin enzymatic hydrolysate to 90° C., keeping the temperature for 10-30 seconds, and then carrying out enzyme deactivation, wherein a hydrolyzing pH is 5.0, and a hydrolyzing temperature is 50° C.; and
step 5, collecting precipitate in the fifth pectin enzymatic hydrolysate obtained in the step 4 after the enzyme deactivation to obtain a pectic polysaccharide, which specifically comprises the following steps of: concentrating the fifth pectin enzymatic hydrolysate once in vacuum under 40-60° C., adding 3-8 times the weight of 95% (v/v) ethanol into a concentrated solution, performing vacuum filtration, collecting solid-phase filter residues, re-dissolving with water, performing vacuum filtration again and dialysis, and sequentially performing vacuum concentration once under 40-60° C. and vacuum freeze-drying to obtain the pectic polysaccharide, wherein in the vacuum freeze-drying, a primary drying temperature is 20° C.-30° C., a secondary drying temperature is 40° C.-50° C., and a cold trap temperature is −50° C.;
wherein, filter paper used in the vacuum filtration is medium-speed qualitative filter paper with an aperture of 8-12 μm, and a vacuum filtration time ranges from 10 seconds to 5 minutes;
the dialysis specifically comprises: dialyzing a filtrate obtained after the vacuum filtration with 0.1 mol/L NaCl as dialysate for 24 hours, replacing the dialysate every 8 hours, dialyzing with deionized water for 24 hours, and replacing the dialysate every 8 hours; during the dialysis, a dialysis temperature is 25° C., and a porosity of a dialysis bag used is 3,000-5,000 Da.

Preferably, in the preparation method of the pectic polysaccharide with the effect of regulating and controlling ice crystal growth, the cellulase is β-1,4-glucan-4-glucan hydrolase.

The present disclosure at least comprises the following beneficial effects.

In the present disclosure, fine structural features of the pectin such as methyl esterification degree, de-esterification region distribution, molecular weight and neutral sugar content (molecular linearity) are adjusted by a precise enzyme digestion modification method, so that neutral sugar side chains causing steric hindrance in the pectin are cut off, methyl ester groups on a main chain are regionalized meanwhile, the obtained pectin methylesterase has the advantage of having a strong adsorption effect with the surface of the ice crystal, which can significantly inhibit the ice crystal growth in the system during freezing.

The pectin methylesterase prepared by the present disclosure can be adsorbed on the surface of an ice core or a small ice crystal through interaction with the ice crystal, which isolates the surface of the ice crystal from water molecules through a masking effect, reduces a mobility of the water molecules, and then makes it difficult to add the water molecules into the ice core, thus achieving the effect of inhibiting the ice crystal growth.

In practical applications, when the pectin is added to a food system or sucrose solution, frozen at −18° C., it can be directly observed that a size of the ice crystal is significantly smaller than that of a group without adding the pectin, and is also significantly smaller than that of a group added with a natural pectin.

The pectin in the modified apple pectin prepared by the present disclosure has a purity higher than 90%, a methyl esterification degree of 45-55%, an acetylation degree less than 2%, a neutral sugar content less than 5%, and an average molecular weight of 1.4-7.5 kDa, and the pectin methylesterase can obviously reduce the ice crystal size in the freezing process.

Other advantages, objects and features of the present disclosure will be embodied in part by the following description, and in part will be understood by those skilled in the art through the study and practice of the present invention.

### BRIEF DESCRIPTION OF THE DRAWINGS

FIG. 1 shows ice crystal morphology of a sucrose solution.

FIG. 2 shows the ice crystal morphology of the sucrose solution added with common commercial apple pectin.

FIG. 3 shows the ice crystal morphology of the sucrose solution added with modified apple pectin polysaccharide according to Embodiment 1 of the present disclosure.

### DESCRIPTION OF THE EMBODIMENTS

The present disclosure will be further described in detail hereinafter with reference to the drawings, so that those skilled in the art can implement the present disclosure with reference to the specification.

It should be understood that terms such as “having,” “including,” and “comprising” as used herein do not exclude the presence or addition of one or more other elements or combinations thereof.

It should be noted that the experimental methods described in the following embodiments, such as those unless otherwise specified, are all conventional methods, and the reagents and materials can be obtained from commercial routes unless otherwise specified.

An idea of the present disclosure is to adjust a molecular weight of a pectin by cutting off a branched neutral sugar part of a pectin molecule, and improve an esterification degree and an ester-based distribution mode of the pectin by using a precise enzymolysis technology, so that the pectin is more easily combined with a surface of an ice crystal to prevent water molecules from participating in ice crystal growth, thereby preparing a pectin with a high ice crystal growth inhibition effect.

In order to enable those skilled in the art to better understand the technical solution of the present disclosure, the following embodiments are provided.

### Embodiment 1

A preparation method of a pectic polysaccharide capable of regulating and controlling ice crystal growth, comprised the following steps:
step 1, extracting from an apple powder to obtain a pectin crude extract: adding water to an apple powder to obtain an apple solution, keeping a temperature of the apple solution at 95° C. for 3 minutes and cooling the apple solution, carrying out enzymolysis with α-amylase, protease, amyloglucosidase and cellulase to obtain an apple solution enzymatic hydrolysate, wherein a concentration of the α-amylase was 495 U/mL, and when enzymolysis with the α-amylase, a pH was 6, a temperature was 62° C., and an enzymolysis time was 1 hour; a concentration of the protease was 60.6 U/mL, and when enzymolysis with the protease, a pH was 7.25, a temperature was 58° C., and an enzymolysis time was 1 hour; a concentration of the amyloglucosidase was 297.5 U/mL, and when enzymolysis with the amyloglucosidase, a pH was 4.0, a temperature was 60° C., and an enzymolysis time was 1 hour; and a concentration of the cellulase was 21.5 U/mL, and when enzymolysis with the cellulose, a pH was 5, a temperature was 50° C., and an enzymolysis time was 2 hours;
keeping the apple solution enzymatic hydrolysate at a temperature of 95° C. for 3 minutes;
concentrating the apple solution enzymatic hydrolysate, and then carrying out the vacuum filtration,
wherein the vacuum filtration treatment comprised the following steps:
adding 5 times the weight of 95% (v/v) ethanol into the concentrated solution of the apple solution enzymatic hydrolysate, stirring uniformly and performing first vacuum filtration, then collecting solid-phase filter residues of the first vacuum filtration and adding ethanol with the same weight as that of the first vacuum filtration, performing second vacuum filtration, collecting solid-phase filter residues of the second vacuum filtration to dissolve in 28 times the weight of acetone, standing for 6 minutes, performing third vacuum filtration, then collecting solid-phase filter residues of the third vacuum filtration for hot acid water extraction to obtain a pectin crude extract, wherein an extraction pH was 2.5, an extraction time was 6 hours, a temperature of water was 70° C., and an acid was 1 mol/L hydrochloric acid;
step 2, carrying out branched chain enzymolysis on the pectin crude extract obtained in the step 1, specially comprising: adding rhamnogalacturonase with a concentration of 5 U/mL into the pectin crude extract, adjusting a pH to be 3.5, subjecting for enzymolysis for 1.5 hours under a condition of 45° C. to obtain a first pectin enzymatic hydrolysate; adding galacturonidase and arabinosidase both with a concentration of 5 U/mL into the first pectin enzymatic hydrolysate, adjusting the pH to be 4.5, and subjecting for enzymolysis for 1.5 hours under a condition of 40° C. to obtain a second pectin enzymatic hydrolysate; and adding xylogalacturonidase with a concentration of 5 U/mL into the second pectin enzymatic hydrolysate, adjusting the pH to be 6.0, and subjecting for enzymolysis continuously for 2 hours under a condition of 50° C. to obtain a third pectin enzymatic hydrolysate, heating the third pectin enzymatic hydrolysate to 90° C., keeping the temperature for 20 seconds, and then carrying out enzyme deactivation;
step 3, adding pectin methylesterase and pectin acetylesterase into the third pectin enzymatic hydrolysate obtained in the step 2 after the enzyme deactivation for precise de-esterification enzymolysis to obtain a fourth pectin enzymatic hydrolysate, heating the fourth pectin enzymatic hydrolysate to 90° C., keeping the temperature for 20 seconds, and then carrying out enzyme deactivation, wherein a dosage of the pectin methylesterase was 1 U/mL, a dosage of the pectin acetylesterase was 0.1 U/mL, a precise de-esterification enzymolysis time was 1.0 hour, the pectin methylesterase was extracted from carrot, a pH value the precise de-esterification enzymolysis was 5.8, and a temperature of the precise de-esterification enzymolysis was 45° C.;
step 4, adding 1.0 U/mL of polygalacturonase into the fourth pectin enzymatic hydrolysate obtained in the step 3 after the enzyme deactivation, and hydrolyzing for 2 hours to obtain a fifth pectin enzymatic hydrolysate to reduce a molecular weight of pectin, and heating the fifth pectin enzymatic hydrolysate to 90° C., keeping the temperature for 20 seconds, and then carrying out enzyme deactivation, wherein a hydrolyzing pH was 5.0, and a hydrolyzing temperature was 50° C.; and
step 5, collecting precipitate in the fifth pectin enzymatic hydrolysate obtained in the step 4 to obtain a pectic polysaccharide after the enzyme deactivation, which specifically comprised the following steps of: concentrating the fifth pectin enzymatic hydrolysate once in vacuum under 40° C., adding 5 times the weight of 95% (v/v) ethanol into a concentrated solution, performing vacuum filtration, collecting solid-phase filter residues, re-dissolving with water, performing vacuum filtration again and dialysis, and sequentially performing vacuum concentration once under 40° C. and vacuum freeze-drying to obtain the pectic polysaccharide, wherein in the vacuum freeze-drying, a primary drying temperature was 25° C., a secondary drying temperature was 45° C., and a cold trap temperature was −50° C.;
wherein, filter paper used in the vacuum filtration was medium-speed qualitative filter paper with an aperture of 10 μm, and a vacuum filtration time was 2 minutes. The dialysis specifically comprised: dialyzing a filtrate obtained after the vacuum filtration with 0.1 mol/L NaCl as dialysate for 24 hours, replacing the dialysate every 8 hours, dialyzing with deionized water for 24 hours, and replacing the dialysate every 8 hours; during the dialysis, a dialysis temperature was 25° C., and a porosity of a dialysis bag used was 3,500 Da.

The pectin in the modified apple pectin prepared by the process according to the embodiment of the present disclosure had a purity higher than 90%, a methyl esterification degree of 52.5%, an acetylation degree of less than 2%, a neutral sugar content of 4.72%, and an average molecular weight of 3.43 kDa.

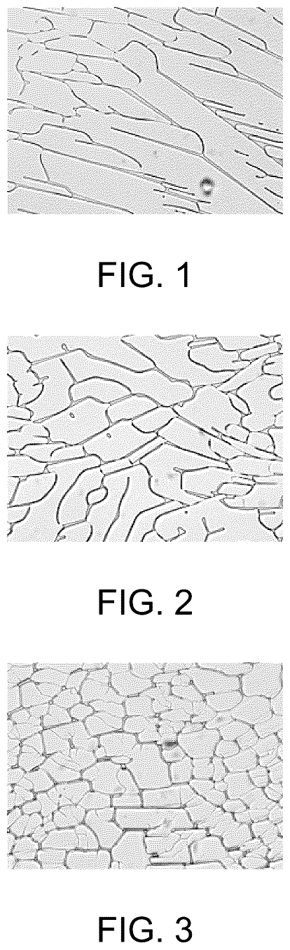

The results of the anti-freezing test show that, as shown in FIG. 3, the pectic polysaccharide prepared in this embodiment can significantly reduce an ice crystal size in a freezing process, effectively reduce the ice crystal size to 1,478 μm², and reduce average ice crystal sizes by 70.9% and 57.5% relative to a group without pectin (FIG. 1) and the group with unmodified pectin (FIG. 2), and an ice crystal inhibiting effect is extremely remarkable. Even compared with a commonly used antifreeze polyethylene glycol, the ice crystal size is relatively reduced by 51.6%. This effect shows that the molecular structure of the pectin plays an important role in inhibiting the ice crystal growth, and the pectin molecule with excellent anti-freezing effect can be obtained through precise structural modification.

### Embodiment 2

A preparation method of a pectic polysaccharide capable of regulating and controlling ice crystal growth, comprised the following steps:
step 1, extracting from an apple powder to obtain a pectin crude extract: adding water to an apple powder to obtain an apple solution, keeping a temperature of the apple solution at 95° C. for 3 minutes and cooling the apple solution, carrying out enzymolysis with α-amylase, protease, amyloglucosidase and cellulase to obtain an apple solution enzymatic hydrolysate, wherein a concentration of the α-amylase was 460 U/mL, and when enzymolysis with the α-amylase, a pH was 5.5, a temperature was 55° C., and an enzymolysis time was 1 hour; a concentration of the protease was 56.7 U/mL, and when enzymolysis with the protease, a pH was 7, a temperature was 50° C., and an enzymolysis time was 1 hour; a concentration of the amyloglucosidase was 285 U/mL, and when enzymolysis with the amyloglucosidase, a pH was 3.8, a temperature was 58° C., and an enzymolysis time was 1 hour; and a concentration of the cellulase was 18 U/mL, and when enzymolysis with the cellulose, a pH was 4.5, a temperature was 45° C., and an enzymolysis time was 2 hours;
keeping the apple solution enzymatic hydrolysate at a temperature of 95° C. for 3 minutes;
concentrating the apple solution enzymatic hydrolysate, and then carrying out the vacuum filtration,
wherein the vacuum filtration treatment comprised the following steps:
adding 5 times the weight of 95% (v/v) ethanol into the concentrated solution of the apple solution enzymatic hydrolysate, stirring uniformly and performing first vacuum filtration, then collecting solid-phase filter residues of the first vacuum filtration and adding ethanol with the same weight as that of the first vacuum filtration, performing second vacuum filtration, collecting solid-phase filter residues of the second vacuum filtration to dissolve in 28 times the weight of acetone, standing for 6 minutes, performing third vacuum filtration, then collecting solid-phase filter residues of the third vacuum filtration for hot acid water extraction to obtain a pectin crude extract, wherein an extraction pH was 2.5, an extraction time was 6 hours, a temperature of water was 70° C., and an acid was 1 mol/L citric acid;
step 2, carrying out branched chain enzymolysis on the pectin crude extract obtained in the step 1, specially comprising: adding rhamnogalacturonase with a concentration of 5 U/mL into the pectin crude extract, adjusting a pH to be 3.5, subjecting for enzymolysis for 1 hour under a condition of 45° C. to obtain a first pectin enzymatic hydrolysate; adding galacturonidase and arabinosidase both with a concentration of 5 U/mL into the first pectin enzymatic hydrolysate, adjusting the pH to be 4.5, and subjecting for enzymolysis for 1 hour under a condition of 40° C. to obtain a second pectin enzymatic hydrolysate; and adding xylogalacturonidase with a concentration of 5 U/mL into the second pectin enzymatic hydrolysate, adjusting the pH to be 6.0, and subjecting for enzymolysis continuously for 2 hours under a condition of 50° C. to obtain a third pectin enzymatic hydrolysate, heating the third pectin enzymatic hydrolysate to 90° C., keeping the temperature for 20 seconds, and then carrying out enzyme deactivation;
step 3, adding pectin methylesterase and pectin acetylesterase into the third pectin enzymatic hydrolysate obtained in the step 2 after the enzyme deactivation for precise de-esterification enzymolysis to obtain a fourth pectin enzymatic hydrolysate, heating the fourth pectin enzymatic hydrolysate to 90° C., keeping the temperature for 20 seconds, and then carrying out enzyme deactivation, wherein a dosage of the pectin methylesterase was 1 U/mL, a dosage of the pectin acetylesterase was 0.1 U/mL, a precise de-esterification enzymolysis time was 1.0 hour, the pectin methylesterase was extracted from carrot, a pH value the precise de-esterification enzymolysis was 5.8, and a temperature of the precise de-esterification enzymolysis was 45° C.;
step 4, adding 2.0 U/mL of polygalacturonase into the fourth pectin enzymatic hydrolysate obtained in the step 3 after the enzyme deactivation, and hydrolyzing for 2.5 hours to obtain a fifth pectin enzymatic hydrolysate to reduce a molecular weight of pectin, and heating the enzymatic hydrolysate to 90° C., keeping the temperature for 20 seconds, and then carrying out enzyme deactivation, wherein a hydrolyzing pH was 5.0, and a hydrolyzing temperature was 50° C.; and step 5, collecting precipitate in the fifth pectin enzymatic hydrolysate obtained in the step 4 after the enzyme deactivation to obtain a pectic polysaccharide, which specifically comprised the following steps of: concentrating the fifth pectin enzymatic hydrolysate once in vacuum under 40° C., adding 5 times the weight of 95% (v/v) ethanol into a concentrated solution, performing vacuum filtration, collecting solid-phase filter residues, re-dissolving with water, performing vacuum filtration again and dialysis, and sequentially performing vacuum concentration once under 40° C. and vacuum freeze-drying to obtain the pectic polysaccharide, wherein in the vacuum freeze-drying, a primary drying temperature was 25° C., a secondary drying temperature was 45° C., and a cold trap temperature was −50° C.;
wherein, filter paper used in the vacuum filtration was medium-speed qualitative filter paper with an aperture of 10 μm, and a vacuum filtration time was 2 minutes. The dialysis specifically comprised: dialyzing a filtrate obtained after the vacuum filtration with 0.1 mol/L NaCl as dialysate for 24 hours, replacing the dialysate every 8 hours, dialyzing with deionized water for 24 hours, and replacing the dialysate every 8 hours; during the dialysis, a dialysis temperature was 25° C., and a porosity of a dialysis bag used was 3,500 Da.

It can be seen from Table 1 that an average molecular weight of the pectin prepared by this process is reduced compared to that of Embodiment 1, which is 1.41 kDa. The too small molecular weight indicates that a polymerization degree of the pectin molecule is relatively low. It can be seen from the embodiment of the present disclosure that the average polymerization degree of the pectin has been reduced to within 10, which reaches a scope of oligosaccharides. Studies have shown that it is desirable to have a specific conformation that can be matched with water molecules on a surface of the ice crystal, while it is also desirable to have a certain steric hindrance to inhibit migration of peripheral water molecules to the surface of the ice crystal after binding. An excessively low molecular weight results in the inability to form an effective ice crystal binding structure, which in turn affects an ability thereof to inhibit the ice crystal.

### Embodiment 3

A preparation method of a pectic polysaccharide capable of regulating and controlling ice crystal growth, comprised the following steps:
step 1, extracting from an apple powder to obtain a pectin crude extract: adding water to an apple powder to obtain an apple solution, keeping a temperature of the apple solution at 95° C. for 3 minutes and cooling the apple solution, carrying out enzymolysis with α-amylase, protease, amyloglucosidase and cellulase to obtain an apple solution enzymatic hydrolysate, wherein a concentration of the α-amylase was 530 U/mL, and when enzymolysis with the α-amylase, a pH was 6.5, a temperature was 70° C., and an enzymolysis time was 1 hour; a concentration of the protease was 64.5 U/mL, and when enzymolysis with the protease, a pH was 7.5, a temperature was 65° C., and an enzymolysis time was 1 hour; a concentration of the amyloglucosidase was 310 U/mL, and when enzymolysis with the amyloglucosidase, a pH was 4.5, a temperature was 65° C., and an enzymolysis time was 1 hour; and a concentration of the cellulase was 25 U/mL, and when enzymolysis with the cellulose, a pH was 5.5, a temperature was 55° C., and an enzymolysis time was 2 hours;
keeping the apple solution enzymatic hydrolysate at a temperature of 95° C. for 3 minutes;
concentrating the apple solution enzymatic hydrolysate, and then carrying out the vacuum filtration,
wherein the vacuum filtration treatment comprised the following steps:
adding 5 times the weight of 95% (v/v) ethanol into the concentrated solution of the apple solution enzymatic hydrolysate, stirring uniformly and performing first vacuum filtration, then collecting solid-phase filter residues of the first vacuum filtration and adding ethanol with the same weight as that of the first vacuum filtration, performing second vacuum filtration, collecting solid-phase filter residues of the second vacuum filtration to dissolve in 28 times the weight of acetone, standing for 6 minutes, performing third vacuum filtration, then collecting solid-phase filter residues of the third vacuum filtration for hot acid water extraction to obtain a pectin crude extract, wherein an extraction pH was 2.5, an extraction time was 6 hours, a temperature of water was 70° C., and an acid was 1 mol/L citric acid;
step 2, carrying out branched chain enzymolysis on the pectin crude extract obtained in the step 1, specially comprising: adding rhamnogalacturonase with a concentration of 5 U/mL into the pectin crude extract, adjusting a pH to be 3.5, subjecting for enzymolysis for 1.5 hours under a condition of 45° C. to obtain a first pectin enzymatic hydrolysate; adding galacturonidase and arabinosidase both with a concentration of 5 U/mL into the first pectin enzymatic hydrolysate, adjusting the pH to be 4.5, and subjecting for enzymolysis for 2 hours under a condition of 40° C. to obtain a second pectin enzymatic hydrolysate; and adding xylogalacturonidase with a concentration of 5 U/mL into the second pectin enzymatic hydrolysate, adjusting the pH to be 6.0, and subjecting for enzymolysis continuously for 2.5 hours under a condition of 50° C. to obtain a third pectin enzymatic hydrolysate, heating the third pectin enzymatic hydrolysate to 90° C., keeping the temperature for 20 seconds, and then carrying out enzyme deactivation;
step 3, adding pectin methylesterase and pectin acetylesterase into the third pectin enzymatic hydrolysate obtained in the step 2 after the enzyme deactivation for precise de-esterification enzymolysis to obtain a fourth pectin enzymatic hydrolysate, heating the fourth pectin enzymatic hydrolysate to 90° C., keeping the temperature for 20 seconds, and then carrying out enzyme deactivation, wherein a dosage of the pectin methylesterase was 1 U/mL, a dosage of the pectin acetylesterase was 0.1 U/mL, a precise de-esterification enzymolysis time was 1.0 hour, the pectin methylesterase was extracted from carrot, a pH value the precise de-esterification enzymolysis was 5.8, and a temperature of the precise de-esterification enzymolysis was 45° C.;
step 4, adding 2.0 U/mL of polygalacturonase into the fourth pectin enzymatic hydrolysate obtained in the step 3 after the enzyme deactivation, and hydrolyzing for 2.5 hours to obtain a fifth pectin enzymatic hydrolysate to reduce a molecular weight of pectin, and heating the fifth pectin enzymatic hydrolysate to 90° C., keeping the temperature for 20 seconds, and then carrying out enzyme deactivation, wherein a hydrolyzing pH was 5.0, and a hydrolyzing temperature was 50° C.; and
step 5, collecting precipitate in the fifth pectin enzymatic hydrolysate obtained in the step 4 after the enzyme deactivation to obtain a pectic polysaccharide, which specifically comprised the following steps of: concentrating the fifth pectin enzymatic hydrolysate once in vacuum under 40° C., adding 5 times the weight of 95% (v/v) ethanol into a concentrated solution, performing vacuum filtration, collecting solid-phase filter residues, re-dissolving with water, performing vacuum filtration again and dialysis, and sequentially performing vacuum concentration once under 40° C. and vacuum freeze-drying to obtain the pectic polysaccharide, wherein in the vacuum freeze-drying, a primary drying temperature was 25° C., a secondary drying temperature was 45° C., and a cold trap temperature was −50° C.;
wherein, filter paper used in the vacuum filtration was medium-speed qualitative filter paper with an aperture of 10 μm, and a vacuum filtration time was 2 minutes. The dialysis specifically comprised: dialyzing a filtrate obtained after the vacuum filtration with 0.1 mol/L NaCl as dialysate for 24 hours, replacing the dialysate every 8 hours, dialyzing with deionized water for 24 hours, and replacing the dialysate every 8 hours; during the dialysis, a dialysis temperature was 25° C., and a porosity of a dialysis bag used was 3,500 Da.

### Embodiment 4

The preparation process was the same as that in Embodiment 1, except that the step 3 of “adding 1.0 U/mL of carrot-derived pectin methylesterase and 0.1 U/mL of pectin acetylesterase into the pectin enzymatic hydrolysate for enzymolysis for 1.0 hour, and quickly inactivating the enzyme and cooling after the reaction” was omitted; in other words, no de-esterification treatment was carried out.

It can be seen from Table 1 that an esterification degree of the pectin molecules prepared by this process is consistent with that of the natural pectin, that is, no de-esterification treatment is carried out. Methyl ester group is a group having a hydrophobic effect, and studies have shown that a hydrophobic group is very critical to an anti-freezing effect of biomacromolecules. In general, most biomacromolecules with anti-freezing activity are amphiphilic substances, groups with hydrophilic conformation are responsible for binding to a surface of the ice crystal, while hydrophobic groups are exposed on the other side to prevent water molecules from getting close to the ice crystal. Hydrophobicity of an acetyl group is higher than that of the methyl ester group, which is very unfavourable for the combination of the pectin molecule and the ice crystal, and acetylation degrees of various embodiments of the present disclosure can be effectively controlled within 2%. However, since the pectin molecule prepared in this solution contains too many methyl ester groups, this indicates that the molecule has fewer galacturonic acid units with free carboxyl groups, which will make it difficult for the molecule to form a sufficient number of regular hydrophilic regions on the whole, which will further affect an ability thereof to bind to the ice crystal and lead to a limited inhibition effect thereof on the ice crystal growth.

### Embodiment 5

The preparation process was the same as that in Embodiment 1, except that the step 3 was only adjusted to “adding 2.0 U/mL of carrot-derived pectin methylesterase and 0.2 U/mL of pectin acetylesterase into the pectin enzymatic hydrolysate for enzymolysis for 2.5 hours, and quickly inactivating the enzyme and cooling after the reaction”, that is, relatively thorough de-esterification treatment was carried out.

It can be seen from Table 1 that the pectin molecule prepared by this process has an esterification degree of 10.6%, and an acetyl group proportion less than 2%, indicating that most of methyl ester groups and acetyl ester groups of the pectin molecules have been hydrolyzed, and most of carboxyl groups on galacturonic acid molecules have been fully exposed. However, when the methyl ester groups are completely hydrolyzed, carboxyl groups on the pectin dominate. Although enough carboxyl groups can form more hydrophilic regions, which is beneficial to the combination with a surface of the ice crystal, too many carboxyl groups will also lead to strong electronegativity and high intermolecular repulsion, which is not conducive to the dense combination with the surface of the ice crystal. On the other hand, too many free carboxyl groups as well as low methyl esterification degree and acetylation degree indicate that the molecules contain less hydrophobic groups, which is not conducive to preventing water molecules from being close to the ice crystal, and in fact will weaken an anti-freezing effect of the molecules.

With reference to Embodiment 1, Embodiment 4 and Embodiment 5, it can be seen that a suitable esterification degree is an important factor for the pectin molecules to have good anti-freezing effect. Through systematic preliminary research, the pectin with an esterification degree of about 50% is screened, which has a good anti-freezing effect.

### Embodiment 6

The preparation process was the same as that in Embodiment 1, except that the pectin methylesterase in the step 3 was not extracted from plants such as carrot, orange peel or tomato, but was replaced with pectin methylesterase extracted from microorganisms such as aspergillus and blackberry.

Generally, plant-derived pectin methylesterase can continuously catalyze the removal of adjacent ester groups on a galacturonic acid chain after binding to the pectin molecule, while the microorganism-derived pectin methylesterase usually hydrolyzes the methyl groups of the pectin in a more random way, so it is difficult to form enough continuous de-esterification regions. It can be seen that although the esterification degree in Embodiment 6 is the same as that in Embodiment 1, a proportion of continuous de-esterified regions is only about 33%. Due to the lack of the continuous de-esterified regions, the pectin cannot form effective hydrophilic regions and hydrophobic domains, which is not conducive to the formation of amphiphilic conformation of the pectin molecule, limiting the combination of the pectin with a surface of the ice crystal, and finally leading to a significant decrease in an ability of the pectin prepared by this scheme to inhibit the ice crystal.

### Embodiment 7

The preparation process was the same as that in Embodiment 1, except that the second step was omitted, that is, the branched structure of the pectin molecule was not hydrolyzed.

It can be seen that the pectin molecules prepared by this process have poor ice crystal inhibition effect, even lower than that of an untreated natural pectin. A proportion of neutral sugar in this pectin is as high as 78.6%. The reason why the neutral sugar molecules are higher than the untreated pectin is that, on one hand, a branched chain structure of the pectin is completely maintained without adding enzymes that hydrolyze pectin branches, which significantly increases a molecular weight of the pectin, and meanwhile forms a huge steric effect, which is not conducive to the combination of the pectin and a surface of the ice crystal. On the other hand, a linear part of the pectin is greatly degraded through a synergistic effect of the pectin methylesterase and polygalacturonase, and an overall effect of keeping a proportion of branched neutral sugar unchanged and reducing a proportion of linear galacturonic acid is to increase a proportion of molecular neutral sugar. With the increase of the proportion of neutral sugar, a proportion of carboxyl groups or strong hydrophilic groups of the pectin also decreases, which makes binding sites of the pectin and the ice crystal significantly reduced, which is extremely unfavorable for the pectin to inhibit the ice crystal.

### Comparative Example 1

For Comparative Example 1, an unmodified natural pectin molecule was added directly to a sucrose solution.

It can be seen that compared with Comparative Example 3 without adding the pectin, the untreated pectin also has a certain ability to inhibit ice crystal growth, but an ability to inhibit the ice crystal growth is significantly lower than that of a common small molecule antifreeze polyethylene glycol.

### Comparative Example 2

For Comparative Example 2, a common small molecule antifreeze polyethylene glycol was added to a sucrose solution. Polyethylene glycol has a significant anti-freezing effect, and it can be seen that an ice crystal size is reduced by about 39.7% as compared to a control group without adding the antifreeze.

### Comparative Example 3

Comparative Example 3 is a control group, which is a group without adding any antifreeze, and it can be seen that an average ice crystal area thereof exceeds 5,000 μm², and an ice crystal size is relatively large.

### Related Analysis Methods:

### Determination of Molecular Weight

A molecular weight of a pectin was determined by using High Performance Size Exclusion Chromatography (HPSEC) combined with multi-angle laser scattering and differential refractive light detector. 5.0 mg of pectin sample was accurately weighed, dissolved in 0.1 mmol/L NaCl solution (mobile phase), and filtered with a 0.22 μm filter membrane, then 200 μL of the mixture was manually injected through quantitative loop at a flow rate of 0.5 mL/min. A weight-average molecular weight (Mw), a number-average molecular weight (Mn) and a polydispersity index (Mw/Mn) of the pectin were calculated and analyzed by using ASTRA 5.3.4 software (Wyatt Technology, Santa Barbara, CA, USA), and a refractive index increment (dn/dc) was set to 0.135 mL/g.

### Determination of Esterification Degree

20.0 mg of pectin was accurately weighed and added with 8 mL of distilled water for ultrasonic treatment for 10 minutes, then added with 3.2 mL of NaOH (2 mmol/L), placed in a constant-temperature oscillation incubator at 20° C. for 1 hour, added with 3.2 mL of HCl (2 mmol/L), neutralized at 25° C. for 15 minutes, and added with a phosphoric acid buffer solution to fix a volume of the mixture to 25 mL. 1.0 mL of hydrolysate was sucked, added with 1.0 mL of ethanol oxidase (1.0 U/mL), subjected to enzymolysis at 25° C. for 15 minutes, then added with 2.0 mL of pentanedione solution, and incubated at 58° C. for 15 minutes. After being cooled and vortex-mixed, absorbance of a standard and the pectin sample was measured at 412 nm by using a UV-1800 ultraviolet spectrophotometer. 633.38 μL of methanol was weighed to fix a volume of the mixture to 50 mL with 0.0975 mmol/L phosphoric acid buffer solution to prepare a stock solution, and a standard curve was made using a methanol standard solution with a concentration gradient of 1-20 μg/mL. A methyl esterification degree was expressed as a mass ratio of methanol to galacturonic acid.

### Proportion Analysis of Branched Neutral Sugar

Content of galacturonic acid (GalA): 5 mg of pectin was weighed, magnetically stirred with 4 mL of concentrated sulfuric acid under an ice bath condition, dropwise added with 2 mL of deionized water, mixed for 5 minutes, dropwise added with 2 mL of deionized water to hydrolyze for 1 hour, and then the mixture was fixed to a volume of 10 mL with deionized water. 0.6 mL of the hydrolyzed sample and galacturonic acid standard solutions with different concentrations were taken in a glass test tube, uniformly vortexed with 1.8 mL of 0.0125 mol/L sulfuric acid-sodium tetraborate solution under an ice water bath condition, subjected to 100° C. oil bath for 5 minutes, cooled in ice water bath, and then vortexed with 60 μL of 3-phenylphenol solution uniformly. The content of GalA in the pectin was scanned by a microplate reader at 520 nm. NaOH solution was used instead of p-phenylphenol solution for a blank control experiment. All experimental samples were conducted in three parallel. The data were expressed in mmolGalA/g pectin.

Composition and content of neutral sugar: the composition of pectin monosaccharide was analyzed by using a high performance anion exchange chromatography system. 10 mg of pectin sample was weighed and fully hydrolyzed with 5 mL of 2 mol/L trifluoroacetic acid at 120° C. for 2 hours. After being cooled to room temperature, the mixture was blown with nitrogen until the solution was completely evaporated. The sample was dissolved with deionized water to fix a volume of the mixture to 10 mL, diluted by 20 times, and then filtered with a 0.22 μm filter membrane. The sample (10 μL) was eluted on a DionexCarboPac PA10 (3×250 mm) column with 250 mmol/L NaOH at a flow rate of 0.5 mL/min. A monosaccharide mixture (0.01-5 mg/L) of fucose (Fuc), rhamnose (Rha), arabinose (Ara), galactose (Gal), glucose (Glc), 169 xylose (Xyl) and GalA were used as qualitative and quantitative standards.

According to the above results, a ratio of a total neutral sugar content to a galacturonic acid content was calculated, which was the proportion of the branched neutral sugar.

### Proportion of Continuous De-Esterified Region

10 mg of natural or pectic polysaccharide sample was accurately weighed, added with galacturonic acid endonuclease first and hydrolyzed at 30° C. for 48 hours, then the hydrolysate was filtered, and contents of galacturonic acid monomer (mono-GalA), dimer (di-GalA) and trimer (tri-GalA) in the sample were analyzed by using a high performance anion chromatography-assisted amperometric detector, and then the proportion of continuous de-esterified region (DB) was calculated according to the following formula based on the measured esterification degree:

$$DB ( \% ) = \frac{[ mono - GalA ] + 2 [ di - GalA ] + 3 [ tri - GalA ]}{( 1 - D M ) \times GalA} \times 100$$

in the formula, DM was the esterification degree and GalA was the content of the galacturonic acid in the molecule.

### Method for Analyzing Ability of Pectin to Inhibit Ice Crystal Growth:

Taking a sucrose solution as a blank system, an effect of adding pectin and other antifreeze substances on an ice recrystallization process was analyzed. A sample solution containing pectic polysaccharide was prepared, with solutions containing natural pectin or polyethylene glycol as control respectively. About 1.5 μL of sample solution was absorbed and dropwise added between two glass slides (Φ12 mm), soaked in oil and sealed, and placed on a cooling table. The sample was rapidly cooled to −40° C. at 30° C./min to induce the formation of multi-ice crystals, and the temperature was raised to −8° C. at 10° C./min and maintained for 30 minutes. During isothermal (−8° C.) annealing, a growth process of the ice crystals was observed and recorded, and N2 was slowly circulated inside and outside the sample table to prevent condensation and atomization of environmental water from affecting an observation field. The experiment was repeated three times independently, and sizes of at least 50 ice crystals in each picture were measured by using ImageJ software, and an average value of the three measurements was taken.

| | | | | Proportion of | Proportion |
|---|---|---|---|---|---|
| | Average ice | Average | | continuous | of branched |
| | crystal area | molecular | Esterification | de-esterified | neutral sugar |
| Item | (μm²) | weight (Da) | degree (%) | region (%) | (%) |
| Embodiment 1 | 1,478 | 3.43 × 10³ | 52.5 | 80.6 | 4.72 |
| Embodiment 2 | 2,042 | 1.41 × 10³ | 52.5 | 80.6 | 4.72 |
| Embodiment 3 | 2,470 | 7.23 × 10³ | 52.5 | 80.6 | 4.72 |
| Embodiment 4 | 3,092 | 3.43 × 10³ | 84.1 | 28.1 | 4.27 |
| Embodiment 5 | 3,157 | 3.43 × 10³ | 10.6 | 91.8 | 4.27 |
| Embodiment 6 | 2,605 | 3.43 × 10³ | 52.5 | 33.1 | 4.27 |
| Embodiment 7 | 3,745 | 4.14 × 10⁵ | 52.5 | 79.3 | 78.6 |
| Comparative | 3,487 | 1.33 × 10⁶ | 84.1 | 25.1 | 51.6 |
| Example 1 | | | | | |
| Comparative | 3,056 | / | / | / | / |
| Example 2 | | | | | |
| Comparative | 5,072 | / | / | / | / |
| Example 3 | | | | | |

The pectin contained in the pectic polysaccharide with high ice crystal growth inhibition effect provided by the present disclosure has a purity higher than 90%, a methyl esterification degree of 45-55%, an acetylation degree less than 2%, a neutral sugar content less than 5%, and an average molecular weight of 1.4-7.5 kDa. Compared with the anti-freezing effect of naturally adding the natural pectin, the anti-freezing pectin polysaccharide of the present disclosure can reduce a size of the ice crystal by more than 50%.

Pectin modification is a systematic project, just like processing a piece of wood, which can be processed into different uses. There are many factors that affect an activity of pectin molecules to inhibit ice crystals, mainly comprising molecular weight, esterification degree, acetylation degree and branching degree. In the present disclosure, pectin methylesterase and polygalacturonase are used for directional deacetylation enzymolysis, which is used to adjust fine structure characteristics of pectin such as methyl esterification degree, de-esterification region, molecular weight and neutral sugar content (molecular linearity), cut off neutral sugar side chains causing steric hindrance in the pectin, and cut off methyl ester groups on the main chain at the same time. The pectin molecules are modified into pectin polysaccharide molecular structure with the best ice crystal inhibiting activity, which has the advantage of strong adsorption on the surface of the ice crystal, and may be used to be added into a food system to inhibit the growth of the ice crystal in the system at low temperature. Improving the inhibitory activity of the ice crystal lies in obtaining pectin polysaccharide with medium molecular weight, less branched chain content and moderate esterification degree. The main structure that finally exerts the activity is the domain of homogalacturonans (HG).

The number and the processing scale of modules described herein are used to simplify the description of the present disclosure. The application, modification and variation of the present disclosure are obvious to those skilled in the art.

Although the implementation of the present disclosure has been disclosed above, it is not limited to the applications listed in the specification and the embodiments, and can be fully applied to various fields suitable for the present disclosure, and additional modifications can be easily implemented by those skilled in the art. Therefore, the present disclosure is not limited to the specific details and embodiments shown and described herein without departing from the general concept defined by the claims and the equivalent scope.

What is claimed is:

1. A preparation method of a pectic polysaccharide capable of regulating and controlling ice crystal growth, comprising the following steps of:
step 1, extracting a pectin crude extract from apple powder: adding water to apple powder to obtain an apple solution, keeping the apple solution at a temperature of 94-97° C. for 1-5 minutes, cooling the apple solution, sequentially carrying out enzymolysis with α-amylase, protease, amyloglucosidase and cellulase to obtain an apple solution enzymatic hydrolysate, wherein a concentration of the α-amylase is 460-530 U/mL, a pH of the α-amylase is 5.5-6.5, a temperature is 55-70° C., and an enzymolysis time is 1 hour; a concentration of the protease is 56.7-64.5 U/mL, a pH of the protease is 7-7.5, a temperature is 50-65° C., and an enzymolysis time is 1 hour; a concentration of the amyloglucosidase is 285-310 U/mL, a pH of the amyloglucosidase during enzymolysis is 3.8-4.5, a temperature is 55-65° C., and an enzymolysis time is 1 hour; and a concentration of the cellulase is 18-25 U/mL, a pH of the cellulase during enzymolysis is 4.5-5.5, a temperature is 45-55° C., and an enzymolysis time is 2 hours;
keeping the apple solution enzymatic hydrolysate at a temperature of 94-97° C. for 1-5 minutes;
concentrating the apple solution enzymatic hydrolysate, and then carrying out the vacuum filtration,
wherein the vacuum filtration treatment comprises the following steps:
adding 3-8 times the weight of 95% (v/v) ethanol into the concentrated solution of the apple solution enzymatic hydrolysate, stirring uniformly and performing first vacuum filtration, then collecting solid-phase filter residues of the first vacuum filtration and adding ethanol with the same weight as that of the first vacuum filtration, performing second vacuum filtration, collecting solid-phase filter residues of the second vacuum filtration to dissolve in 25-30 times the weight of acetone, standing for 4-8 minutes, performing third vacuum filtration, then collecting solid-phase filter residues of the third vacuum filtration for hot acid water extraction to obtain a pectin crude extract, wherein an extraction pH is 2-3, an extraction time is 2-10 hours, a temperature of water is 65° C.-90° C., and an acid is 1 mol/L hydrochloric acid or 1 mol/L citric acid;
step 2, carrying out branched chain enzymolysis on the pectin crude extract obtained in the step 1, specially comprising: adding rhamnogalacturonase with a concentration of 2-5 U/mL into the pectin crude extract, adjusting a pH to be 3.5, subjecting for enzymolysis for 1-1.5 hours under a condition of 45° C. to obtain a first pectin enzymatic hydrolysate; adding galacturonidase and arabinosidase both with a concentration of 2-5 U/mL into the first pectin enzymatic hydrolysate, adjusting the pH to be 4.5, and subjecting for enzymolysis for 1-2 hours under a condition of 40° C. to obtain a second pectin enzymatic hydrolysate; and adding xylogalacturonidase with a concentration of 2-5 U/mL into the second pectin enzymatic hydrolysate, adjusting the pH to be 6.0, and subjecting for enzymolysis continuously for 2-2.5 hours under a condition of 50° C. to obtain a third pectin enzymatic hydrolysate, heating the third pectin enzymatic hydrolysate to 90° C., keeping the temperature for 10-30 seconds, and then carrying out enzyme deactivation;
step 3, adding pectin methylesterase and pectin acetylesterase into the third pectin enzymatic hydrolysate obtained in the step 2 after the enzyme deactivation for precise de-esterification enzymolysis to obtain a fourth pectin enzymatic hydrolysate, heating the fourth pectin enzymatic hydrolysate to 90° C., keeping the temperature for 10-30 seconds, and then carrying out enzyme deactivation, wherein a dosage of the pectin methylesterase is 0.2-2 U/mL, a dosage of the pectin acetylesterase is 0.1-0.2 U/mL, a precise de-esterification enzymolysis time is 1.0-2.5 hours, the pectin methylesterase is extracted from tomato, orange, pawpaw, broccoli, apple or carrot, a pH value of the precise de-esterification enzymolysis is 5.8, and a temperature of the precise de-esterification enzymolysis is 45° C.;
step 4, adding 0.5-2 U/mL of polygalacturonase into the fourth pectin enzymatic hydrolysate obtained in the step 3 after the enzyme deactivation, and hydrolyzing for 1.5-2.5 hours to obtain a fifth pectin enzymatic hydrolysate to reduce a molecular weight of pectin, and heating the fifth pectin enzymatic hydrolysate to 90° C., keeping the temperature for 10-30 seconds, and then carrying out enzyme deactivation, wherein a hydrolyzing pH is 5.0, and a hydrolyzing temperature is 50° C.; and
step 5, collecting precipitate in the fifth pectin enzymatic hydrolysate obtained in the step 4 after the enzyme deactivation to obtain a pectic polysaccharide, which specifically comprises the following steps of: concentrating the fifth pectin enzymatic hydrolysate once in vacuum under 40-60° C., adding 3-8 times the weight of 95% (v/v) ethanol into a concentrated solution, performing vacuum filtration, collecting solid-phase filter residues, re-dissolving with water, performing vacuum filtration again and dialysis, and sequentially performing vacuum concentration once under 40-60° C. and vacuum freeze-drying to obtain the pectic polysaccharide, wherein in the vacuum freeze-drying, a primary drying temperature is 20° C.-30° C., a secondary drying temperature is 40° C.-50° C., and a cold trap temperature is −50° C.;
wherein, filter paper used in the vacuum filtration is medium-speed qualitative filter paper with an aperture of 8-12 μm, and a vacuum filtration time ranges from 10 seconds to 5 minutes;
the dialysis specifically comprises: dialyzing a filtrate obtained after the vacuum filtration with 0.1 mol/L NaCl as dialysate for 24 hours, replacing the dialysate every 8 hours, dialyzing with deionized water for 24 hours, and replacing the dialysate every 8 hours; during the dialysis, a dialysis temperature is 25° C., and a porosity of a dialysis bag used is 3,000-5,000 Da.

2. The preparation method of the pectic polysaccharide capable of regulating and controlling ice crystal growth according to claim 1, wherein the cellulase is β-1,4-glucan-4-glucan hydrolase.
